(19) United States
(12) Patent Application Publication
(10) Pub. No.: US 2002/0018759 A1
(43) Pub. Date: Feb. 14, 2002

(54) Nail enamel compositions, related methods, and a two component kit for painting the nails

(76) Inventors: Frank Charles Pagano, Avenel, NJ (US); George Harvey Armstrong, Dayton, NJ (US); Anjali Abhimanyu Patil, Westfield, NJ (US); Joseph Frank Calello, Bridgewater, NJ (US); Robert Walter Sandewicz, Monroe Township, NJ (US)

Correspondence Address: Julie Blackburn, Revlon Consumer Products Corporation, 625 Madison Avenue, New York, NY, 10022, US

(21) Appl. No.: 09/843,000

(22) Filed: Apr. 26, 2001

Related U.S. Application Data
(60) Provisional application No. 60/202,106, filed on May 4, 2000.

(51) Int. Cl. A61K 7/04

(52) U.S. Cl. 424/61; 523/105

(57) ABSTRACT

A nail enamel composition comprising, by weight of the total composition, about 10-95% solvent, and 5-95% of a film forming polymer having a glass transition temperature in the range of 5 to 90° Centigrade, and containing about 2 to 29% by weight of the total polymer of at least one polar monomer; a two container kit for polishing the nails containing the nail enamel composition of the invention and a second composition comprising 1-99% solvent and 1-99% of an film forming polymer; and method for polishing nails using the composition and kit of the invention.

This application claims priority from provisional application Ser. No. 60/202,106 filed May 4, 2000.

### TECHNICAL FIELD

The invention is in the field of compositions for application to fingernails and toenails.

### BACKGROUND OF THE INVENTION

Traditional nail enamel compositions generally comprise a film former, a plasticizer, and a solvent. Cellulose derivatives, and in particular nitrocellulose, is most commonly used as a film former in commercial nail enamels because it is inexpensive and readily available. In addition, nail enamels containing cellulose-based film formers tend to provide good wear, adhesion, and gloss. However, nitrocellulose has certain undesirable features. For example, it is essentially gun cotton, an explosive, so its manufacture and transport prior to incorporation into nail enamel poses certain hazards. Moreover, in some cases nitrocellulose may yellow in the nail enamel as it ages.

Nail enamels based upon polymeric film formers are known in the art. Some of these nail enamels do not contain nitrocellulose, or contain it in much smaller amounts. However, in many cases, the polymeric film formers do not provide the wear, adhesion, and gloss which is desired for commercial preparations. Thus, the goal for cosmetics companies is to develop nail enamels based upon polymeric systems (preferably without nitrocellulose, or containing reduced amounts of nitrocellulose) which provide superior gloss, adhesion, and wear when compared to the currently available products.

One object of the invention is to provide a polymer-based nail enamel composition which provides good wear, adhesion, and gloss.

Another object of the invention is to provide a polymer-based nail enamel composition which can be made either without cellulose-based film formers, or containing significantly reduced levels of cellulose-based film formers.

Another object of the invention is to provide a method for forming a film on nails which is more resistant to wear when compared with normal methods and preparations.

Another object of the invention is to provide nail enamel compositions that exhibit reduced yellowing of the nails.

### BACKGROUND OF THE INVENTION

The invention is directed to a nail enamel composition comprising, by weight of the total composition:

10-95% solvent, and

5-95% of a polymer having a glass transition temperature in the range of 5 to 90° C., and containing about 2 to 29% by weight of the total polymer of at least one polar monomer.

The invention is further directed to a two container kit for polishing nails comprising:

(a) a first container containing a nail enamel composition comprising, by weight of the total composition:

10-95% solvent, and

5-95% of a film forming polymer having a glass transition temperature in the range of 5 to 90° C. and containing 2 to 29% by weight of the total polymer of at least one polar monomer; and

(b) a second container containing a nail enamel topcoat composition comprising, by weight of the total topcoat composition:

1-99% solvent, and

1-99% of a film former.

The invention is further directed to a method for polishing the nails comprising:

(a) applying to the nails a first composition comprising, by weight of the total composition:

10-95% solvent, and

5-95% of a film forming polymer having a glass transition temperature in the range of 5 to 90° C. and containing about 2 to 29% by weight of the total polymer of at least one polar monomer;

(b) applying to the nails a second composition comprising, by weight of the total composition:

1-99% solvent, and

1-99% of a film former.

### DETAILED DESCRIPTION

All percentages mentioned herein are percentages by weight unless otherwise indicated.

I. The Nail Enamel Composition

The nail enamel composition of the invention comprises, by weight of the total composition, about 10-95% solvent and about 5-95% of a film forming polymer having a glass transition temperature in the range of 5 to 90, preferably 10 to 55° C., and containing about 2 to 29%, preferably about 2 to 25% by weight of the total polymer, of at least one polar monomer.

A. The Solvent

The nail enamel composition comprises 10-95%, preferably 15-90%, more preferably 20-80% of a solvent. The solvent may be aqueous or non-aqueous or a mixture of both types of solvents. Suitable non-aqueous solvents include aliphatic or aromatic ketones such as acetone, diacetone alcohol, dihydroxyacetone, ethyl butyl valerolactone, methyl ethyl ketone, and the like; aliphatic or aromatic alcohols such as methanol, propanol, benzyl alcohol, butoxyethanol, butoxypropanol, butyl alcohol, 3-methyl-3-methoxy-butanol, t-butyl alcohol, butylene glycol, diethylene glycol, abietyl alcohol, propylene carbonate, hexyl alcohol, isopropanol, and the like; glycol ethers; esters such as butyl acetate, ethyl acetate 1-methoxy-2-propanol acetate; benzoates and the like.

B. The Film Forming Polymer

The film forming polymer used in the nail enamel compositions is comprised of at least two monomers, and has a glass transition temperature in the range of 5 to 90, preferably 10 to 55° C. The film forming polymer contains about 2-29%, preferably 5-25%, more preferably 8-12% by weight of the total polymer of at least one polar monomer; with the remainder of the film forming polymer being comprised of one or more nonpolar monomers. It is important that the amount of polar monomer in the film forming polymer is preferably within the range of 2 to 20% by weight of the total polymer composition.

The term “film forming polymer” means a polymer that is soluble or dispersible in the solvent of the nail enamel composition and is capable of remaining soluble or dispersible, without settling out, in the composition, during the shelf life of the product. At the same time, when the composition is applied to the nails and the solvent dries, the polymer should be capable of forming a permanent, adherent film on the nails having a thickness ranging from about 0.5 to 8 mils (thousandths of an inch). The film forming polymer is further characterized in that once it is applied to nails in the form of a nail enamel composition, the film cannot be removed with water alone, but rather is removable only with organic solvent-based removers containing ingredients such as acetone, butyl acetate, ethyl acetate, methyl ethyl ketone, and the like.

The term “glass transition temperature” means the temperature at which an amorphous material such as glass or a high polymer changes from a brittle, vitreous state to a plastic state. With respect to acrylics and acrylates, the glass transition temperature generally correlates with the number of carbon atoms in the ester group, i.e. the greater the number of carbon atoms the lower the glass transition temperature. The glass transition temperature of a polymer may be theoretically calculated according to the following formula:

$$\frac{1}{Tg} = \frac{{W}_{1}}{{Tg}_{1}} + \frac{{W}_{2}}{{Tg}_{2}} + \frac{{W}_{3}}{{Tg}_{3}} + … \frac{{W}_{n}}{{Tg}_{n}}$$

where Tg is the glass transition temperature of the polymer in degrees Kelvin; W₁, W₂, W₃ . . . Wₙ are the weight fractions of each of the components of the polymer, and Tg₁, Tg₂, Tg₃, and Tgₙ are the Tg in degrees Kelvin, of the homopolymer [Reference: T. G. Fox, Bull. Am. Phys. Soc., No. 3, page 123 (1956)]. A more practical way to measure Tg is by differential scanning calorimetry (DSC) which measures the change in enthalpy of a polymer with temperature. Preferably, the Tg polymers used in the compositions of the invention are measured by DSC. It is important that the glass transition temperature of the polymer used in the nail enamel compositions be within the specified range. Such polymers provide suitable hardness to the adherent film as well as suitable plasticity. Polymers having glass transition temperatures outside the range of 5 to 90° C. are either too hard or too soft. For example, a polymer having a Tg that is more than 90° C. will be too hard and exhibit reduced plasticity, such that when the polymer is applied to the nail in the form of a nail enamel composition, the nail enamel provides a film that is too brittle, causing the enamel to chip too easily. On the other hand, if the glass transition temperature of the polymer is below 5° C., the polymer exhibits inadequate hardness, and in certain cases, too much plasticity. It should also be understood that the glass transition temperature of the different monomer repeat units found within the polymer may fall outside the range of 5 to 90° C., so long as the combined glass transition temperature of the individual monomer repeat units falls within the range. In particular, the polymers may contain hard and soft segments or portions. By “hard” is meant that a portion of the polymer has repeat units having a glass transition temperature falling within the range of 50-100° C. By “soft” is meant a portion of the polymer has a glass transition temperature falling within the range of −10 to 49° C. For example, in a copolymer of methyl methacrylate and butyl methacrylate, the methyl methacrylate portion is “hard”, methyl methacrylate having a glass transition temperature in the range of 50-100° C. The butyl methacrylate portion is “soft”, butyl methacrylate having a glass transition temperature in the range of −10-49° C. The polymer may be comprised of any mixture of soft and hard portions, and those soft and hard portions individually may have a glass transition temperature which does not fall within the range of 5 to 90° C., however, the combined glass transition temperature of all of the polymer portions must fall within the range of 5 to 90° C.

1. The Polar Monomer

The film forming polymer used in the compositions of the invention comprises 2-29, preferably 2-25%, more preferably 5-14% by weight of the total polymer, of one or more polar monomers. The term “polar monomer” means a monomer having a hydrophilic functional group which confers charge, or polarity, to the monomer. Examples of hydrophilic functional groups that may be found on the polar monomer include hydroxy-polyethyleneoxy, hydroxyl, carboxylates such as carboxylic acid, sulfonates, sulfates, amines, or phosphates. These polar, hydrophilic functional groups may be anionic or cationic in charge. For example, hydroxy-polethyleneoxy, hydroxyl, carboxylates, sulfonates, phosphates, and sulfates tend to be anionic in charge, whereas most amines or amides will be cationically charged. Thus, if the amount of polar monomer in the polymer is more than 29% by weight of the total polymer, the polymer becomes water sensitive and the nail enamel composition is too easily removed from the nail. On the other hand, if the amount of polar monomer in the polymer is less than 2% by weight of the total polymer, the nail enamel composition does not exhibit any improvement in adhesion to the nail.

A variety of polar monomers are suitable, in particular, ethylenically unsaturated monomers such as acrylic acid esters or methacrylic acid esters substituted with one or more hydroxyl groups or hydroxy polyethylene-oxy groups; monomers containing carboxylic acid functional groups such as acrylic acid, methacrylic acid, and the like, amine functional acrylates and methacrylates, and so on. Preferably the polar monomer has the general formula:

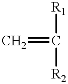

wherein R₁ is H, or a C₁₋₃₀ straight or branched chain alkyl, aryl, or aralkyl; and R₂ is COOM wherein M is H; (CHR₁)ₙOH; (CH₂CH₂O)ₙH, (CH₂)ₙNR₁; (CHR₁CONR₁H) where n is 1-100.

Preferably, the polar monomer is acrylic acid or methacrylic acid, more preferably acrylic acid.

2. The Nonpolar Monomer

The film forming polymer used in the nail enamel compositions of the invention comprises 90-98%, preferably 75-95%, more preferably 85-95% by weight of the total polymer of at least one nonpolar monomer. A wide variety of nonpolar monomers may be used, and the polymer may be comprised more than one nonpolar monomer, provided that the amount of nonpolar monomer in the total polymer composition is between about 75 to 98%. In general, the nonpolar monomer is an ethylenically unsaturated monomer that is mono-, di-, tri-, or polyfunctional as regards the addition-polymerizable ethylenic bonds. A variety of ethylenically unsaturated monomers are suitable, so long as the monomers selected are nonpolar and capable of polymerization with the polar monomer to form a film forming polymer that is soluble or dispersible in the solvent of the nail enamel composition and which forms an adherent, permanent, non-water removable film on the nail after the nail enamel composition is applied to the nail and allowed to dry.

(a) Monofunctional Monomers

Examples of suitable monofunctional ethylenically unsaturated monomers include those of the formula:

I.

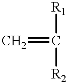

wherein R₁ is H, a C₁₋₃₀ straight or branched chain alkyl, aryl, aralkyl; R₂ is H, CH₃, a pyrrolidone, or a substituted or unsubstituted aromatic, alicyclic, or bicyclic ring where the substitutents are C₁₋₃₀ straight or branched chain alkyl, or COOM wherein M is a C₁₋₃₀ straight or branched chain alkyl, pyrrolidone, or a substituted or unsubstituted aromatic, alicylic, or bicyclic ring where the substitutents are C₁₋₃₀ straight or branched chain alkyl which may be substituted with one or more halogens.

Preferably, the monofunctional ethylenically unsaturated monomer is of Formula I, above, wherein R₁ is H or a C₁₋₃₀ alkyl, and R₂ is COOM wherein M is a C₁₋₃₀ straight or branched chain alkyl.

More preferably, R₁ is H or CH₃, and R₂ is COOM wherein M is a C₁₋₁₀ straight or branched chain alkyl. In the preferred embodiment of the invention, the monofunctional ethylenically unsaturated monomer comprises the monomer of Formula I wherein R₁ is CH₃ and R₂ is COOM where M is a C₄ alkyl, in particular butyl, and the monomer is butyl methacrylate.

Di-, tri- and polyfunctional monomers, as well as oligomers, of the above monofunctional monomers may also be used in the composition. Such di-, tri-, and polyfunctional monomers are generally known as cross-linking monomers because they aid in cross-linking of the monomer composition during and after polymerization.

(b) Difunctional Monomers

Preferred difunctional monomers include those having the general formula:

II.

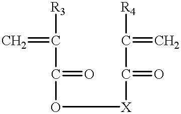

wherein R₃ and R₄ are each independently H, a C₁₋₃₀ straight or branched chain alkyl, aryl, or aralkyl; and X is [(CH₂)ₓO_{y}]_{z} wherein x is 3-20, and y is 1, and z is 1-100. Particularly preferred are difunctional acrylates and methacrylates, such as the compound of formula II above wherein R₃ and R₄ are CH₃ and X is [(CH₂)ₓO_{y}]_{z} wherein x is 1-4; and y is 1-20; and z is 1-10.

Particularly preferred are difunctional acrylates and methacrylates, such as the compound of formula II above wherein R₃ and R₄ are CH₃ and X is [(CH₂)ₓO_{y}]_{z} wherein x is 3; and y is 1, and z is 4. Examples of difunctional monomers include propylene glycol dimethacrylate or dipropylene glycol dimethacrylate.

Trifunctional and polyfunctional monomers are also suitable for use in the polymerizable monomer compositions of the invention. Examples of such monomers include acrylates and methacrylates such as trimethylolpropane trimethacrylate or trimethylolpropane triacrylate.

C. Other Ingredients

The nail enamel compositions of the invention may contain one or more additional ingredients such as pigments, suspending agents, defoaming agents, secondary film formers, plasticizers, and so on.

(a) Pigments

The nail enamel compositions of the invention may be pigmented or clear. If pigmented, generally 0.1-30% by weight of the total composition, preferably 0.5-20%, more preferably 1-15% of pigment is suggested. Pigments suitable for use in nail enamel compositions are well known and include iron oxides, D&C and FD&C colors, titanium dioxide, and the like. The pigments may be treated or coated with agents which modify the surface properties such as silicones. Examples of silicone treated pigments which can be used in the compositions of the invention are set forth in U.S. Pat. No. 4,832,944, which is hereby incorporated by reference.

(b) Suspending Agents

If the nail enamel compositions of the invention contain pigments, it is desirable to also incorporate 0.01-15%, preferably 0.05-10%, more preferably 0.1-8% by weight of the total composition of a suspending agent which acts to suspend the pigments in the formulation. Suitable suspending agents are montmorillonite minerals and derivatives thereof, such as stearalkonium bentonite, hectorites, attapulgite, bentones, and the like, as well as polymeric compounds known as associative thickeners. Suitable associative thickeners generally contain a hydrophilic backbone and hydrophobic side groups. Examples of such thickeners include polyacrylates with hydrophobic side groups, cellulose ethers with hydrophobic side groups, polyurethane thickeners. Examples of hydrophobic side groups are long chain alkyl groups such as dodecyl, hexadecyl, or octadecyl; alkylaryl groups such as octylphenyl or nonyphenyl.

(c) Defoaming Agents

Suitable defoaming agents for use in the composition include silicone glycol copolymers. Suggested ranges are 0.01-10%, preferably 0.05-8%, more preferably 0.1-5% by weight of the total composition. Silicone glycol copolymers which may be used in the compositions of the invention are polymethylsiloxanes wherein a portion of the methylsiloxane units are substituted with polyalkylene glycol ether moieities. Preferred is wherein about 60-90% of the polymer (the percentage being based on the number of monomer units), of the compound is polydimethylsiloxane or polyhydrogen methylsiloxane and 30-40% of the compound (the percentage being based upon the number of monomer units) is di-methyl or hydrogen-methyl siloxane units substituted with polyalkylene glycol ethers. Most preferred are silicone glycol copolymers having a viscosity ranging from 1.0 to 500,000, preferably 1.0 to 2,000 centipoise at 25° C., a specific gravity ranging from 0.80 to 1.030 at 25° C., and comprise approximately 80% dimethylsiloxane units and 20% propylene oxide substituted methyl siloxane units. Silicone glycol copolymers having this description are commercially available from a variety of sources including Dow Corning under the tradenames Dow Corning Additive 3, 7, 11, 14, 18, 21, 24, 26, 28, 29, 51, 54, 56, 57, and 1248.

(d) Secondary Film Formers

It may be desirable to add small levels of secondary film formers such as cellulosic film formers. Suitable cellulosic film formers include nitrocellulose, cellulose acetate isobutyrate, cellulose acetate propionate, and the like. If cellulosic film formers are added, a level of 0.1-25%, preferably 0.5-10%, more preferably 0.5-5% by weight of the total composition is suggested.

(e) Plasticizers

Preferably, the nail enamel compositions contain one more plasticizers that plasticize the film formed on the nail after the composition is applied to the nail and allowed to try. Suggested ranges of plasticizer are 0.1-35%, preferably 0.5-30%, more preferably 1-25% by weight of the total composition. Suitable plasticizers include glyceryl, glycol, and citrate esters as disclosed in U.S. Pat. No. 5,066,484, which is hereby incorporated by reference. Examples of such esters include glyceryl tribenzoate, glyceryl triacetate, acetyl tributyl citrate, dipropylene glycol dibenzoate, and the like. Also suitable, are plasticizers of the following general formula:

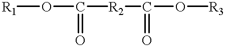

wherein R₁, R₂, and R₃ are each independently a C₁₋₂₀ straight or branched chain alkyl or alkylene which may be substituted with one or more hydroxyl groups. Preferably, R₁ is a C₃₋₁₀ straight or branched chain alkyl; R₂ is a C₂₋₈ alkyl which may be substituted with one or more hydroxyl groups; and R₃ is a C₃₋₁₀ straight or branched chain alkyl. Examples of such compounds include dioctyl malate, diisopropyl adipate, dibutyl sebacate, dioactyl azelate, dioctyl succinate, dioctyl fumarate, and the like. Preferred is where R₁ and R₃ are a branched C₈alkyl, R₂ is a C₂ alkyl substituted with one hydroxy group, which is dioctyl malate.

II. The Two Container Kit

The invention is further directed to a two container kit for polishing nails comprising:

(a) a first container containing a nail enamel composition comprising, by weight of the total composition:

10-95% solvent, and

5-95% of a film forming polymer having a glass transition temperature in the range of 5 to 90° C. and containing 2 to 29% by weight of the total polymer of at least one polar monomer; and

(b) a second container containing a nail enamel topcoat composition comprising, by weight of the total topcoat composition:

1-99% solvent, and

1-99% of a film forming polymer.

The nail enamel composition described in Section I is found in one container within the two container kit. The second container contains a nail enamel composition comprising 1-99% of a solvent which can be aqueous or non-aqueous. Suitable solvents include those set forth in Section I.A, above.

In addition the nail enamel of the second container comprises at least one film forming polymer that is preferably not pigmented, e.g. is clear or translucent in the nail enamel composition. In addition, the film forming polymer may be tinted, which means that it is dyed by a water or solvent soluble organic dye. Examples of suitable clear polymers include cellulosic basic film formers as described in Section I.C.(d), above. Also suitable are polymers made from ethylenically unsaturated nonpolar monomers as described in Section I.B.2, above. Preferably the second container comprises a cellulosic based film former in combination with a film forming comprised of polymerized ethylenically unsaturated monomers which are acrylate or methacrylate based.

III. The Method

The invention further comprises a method for polishing the nails comprising:

(a) applying to the nails a first composition comprising, by weight of the total composition:

10-95% solvent, and

5-95% of a film forming polymer having a glass transition temperature in the range of 5 to 90° C. and containing about 2 to 29% by weight of the total polymer of at least one polar monomer;

(b) applying to the nails a second composition comprising, by weight of the total composition:

1-99% solvent, and

1-99% of a film former.

The first composition may be applied directly to the nail, or applied to the nail after a basecoat. Suitable basecoats are well known in the art, and usually comprise clear nail enamel compositions containing a film former and solvent. Preferably, the first composition is applied directly to the nail without use of a basecoat. It is believed that applying the first composition directly to the nail promotes adhesion to the nail. Either one or two coats of this composition may be applied to the nail, after which the composition is allowed to dry for a period of time ranging from 1 second to several minutes. After drying of the first composition, a second composition is applied to the nail as a topcoat and it is allowed to dry.

It has been discovered that this two step system provides substantially improved wear. The term “wear” indicates the overall resistance of the dried nail enamel film to chipping, loss of gloss, scratching, or other reduction in aesthetics due to tackiness of the coating or sensitivity of the coating to heat and moisture when on the nails.

The invention will be further described in connection with the following examples which are set forth for the purposes of illustration only.

### EXAMPLE 1

A nail enamel composition in accordance with the invention was made according to the following formula:

| | w/w % |
|---|---|
| Polymer solution¹ | 63.00 |
| Dipropylene glycol dibenzoate | 5.20 |
| Suspending agent² | 14.00 |
| Dimethicone copolyol | 0.20 |
| 1-methoxy-2-propanol | 5.00 |
| Butyl acetate | 8.60 |
| Color pigments | 4.20 |

¹a solution comprising about 41% by weight of the solution of a copolymer containing 90 parts by weight of butyl methacrylate and 10 parts by weight of acrylic acid, having a molecular weight of about 68,000; and about 59% by weight of the solution of 80 parts by weight of ethyl acetate and 20 parts by weight of butyl acetate.
²a mixture of 7% by weight of the total mixture of stearalkonium bentonite, 17.6% by weight of nitrocellulose, 65.5% by weight of butyl acetate, and 9.9% by weight of isopropanol.

### EXAMPLE 2

Nail enamel compositions in accordance with the invention were prepared as follows:

w/w %

| | 1 | 2 |
|---|---|---|
| Polymer solution¹ | 54.1 | 65.2 |
| Dioctyl malate | 6.3 | 5.4 |
| Suspending agent² | 13.0 | 14.0 |
| Ethyl acetate | 8.2 | — |
| Butyl acetate | 9.7 | 6.0 |
| Methoxypropanol | 4.4 | — |
| 1-methoxy-2-propanol acetate | — | 5.2 |
| Pigment | 4.3 | 4.2 |

¹a solution comprising about 41% by weight of the solution of a copolymer containing 90 parts by weight of butyl methacrylate and 10 parts by weight of acrylic acid, having a molecular weight of about 68,000; and about 59% by weight of the solution of 80 parts by weight of ethyl acetate and 20 parts by weight of butyl acetate.
²a mixture of 7% by weight of the total mixture of stearalkonium bentonite, 17.6% by weight of nitrocellulose, 65.5% by weight of butyl acetate, and 9.9% by weight of isopropanol.

The compositions were made by combining the ingredients and mixing well. The compositions were poured into glass nail enamel containers.

### EXAMPLE 3

A nail enamel topcoat composition for use in the second container of the two container kit, was prepared as follows:

| | w/w % |
|---|---|
| Butyl acetate | 23.90 |
| Ethyl acetate | 38.61 |
| Nitrocellulose | 15.69 |
| Glyceryl tribenzoate | 8.20 |
| N-butyl alcohol | 0.50 |
| Acrylates copolymer/butyl acetate (50:50) | 10.00 |
| Triacetin | 1.00 |
| Ethyl acetate/etocrylene solution (75:25) | 2.00 |
| Butyl acetate/dimethicone (95:5) | 0.10 |

The composition was made by combining all of the ingredients and mixing well. The composition was poured into glass nail enamel containers.

### EXAMPLE 4

Side-by-side evaluations of the nail enamel compositions and method of the invention and classic Revlon nail enamel were conducted to ascertain differences in wear. A two pack nail enamel system was prepared by filling a first container with the nail enamel composition of Example 1 (“Container 1”). A second container was filled with the nail enamel composition of Example 3 (“Container 2”). A third container was filled with the nail enamel composition set forth below (“Container 3 ”):

Fourteen test subjects participated in this double blind study. A two pack nail enamel system comprising Containers 1 and 2 was applied to the fingernails on one hand of each subject. In particular, the composition of Container 1 was applied to the nails and allowed to dry for approximately 60 seconds. Then, the nail enamel composition of Container 2 was applied over the dried Container 1 film and allowed to dry. The nail enamel composition of Container 3 was applied to the nails on the other hand of each subject. The composition was allowed to dry.

### EXAMPLE 5

A two pack nail enamel system was prepared by filling a first container with the nail enamel composition of Example 1 (“Container 1”). A second container was filled with the nail enamel composition of Example 3 (“Container 2”). A third container was filled with a commerically available Classic Revlon Nail Enamel (“Container 3”):

Seven test subjects participated in this double blind study. A two pack nail enamel system comprising Containers 1 and 2 was applied to the fingernails on the alternate fingers on one hand of each subject. In particular, the composition of Container 1 was applied to the nails and allowed to dry. Then, the nail enamel composition of Container 2 was applied over the dried Container 1 film and allowed to dry. The nail enamel composition of Container 3 was applied to the nails on the other hand of each subject.

The wear was rated on a scale of 1 to 10 where 1 is the best and 10 is the worst.

| | 2 Pack (wear) | |
|---|---|---|
| Day | Container 1 + Container 2 | Container 3 (wear) |
| 2 | 1 | 1 |
| 3 | 1 | 1 |
| 4 | 1 | 2 |
| 5 | 1 | 2 |
| 8 | 2 | 5 |
| 9 | 3 | 5 |
| 10 | 3 | 6 |

The above results show that the nail enamel compositions of the invention provide substantially improved wear when compared to a currently available nail enamel composition.

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A nail enamel composition comprising, by weight of the total composition:
10-95% solvent, and
5-95% of a polymer having a glass transition temperature in the range of 5 to 90° C., and containing about 2 to 29% by weight of the total polymer of at least one polar monomer.

2. The composition of claim 1 wherein the solvent is aqueous.

3. The composition of claim 1 wherein the comprises a non-aqueous solvent.

4. The composition of claim 3 wherein the non-aqueous solvent is an aliphatic or aromatic ketone; aliphatic or aromatic alcohol; glycol ether; ester, or mixtures thereof.

5. The composition of claim 1 wherein the film forming polymer the polar monomer is anionically or cationically charged.

6. The composition of claim 5 wherein the polar monomer is anionically charged.

7. The composition of claim 6 wherein the polar monomer has the general formula: 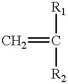
wherein R₁ is H, or a C₁₋₃₀ straight or branched chain alkyl, aryl, or aralkyl; and R₂ is COOM wherein M is H; (CR₁)ₙOH; (CH₂CH₂O)ₙH,(CH₂)ₙNR₁; where n is 1-100.

8. The composition of claim 7 wherein the polar monomer, R₁ is H or CH₃, and R₂ is COOM wherein M is H.

9. The composition of claim 8 wherein the polar monomer is acrylic acid.

10. The composition of claim 1 further comprising 0.1-30% by weight of the total composition of pigment.

11. The composition of claim 1 further comprising 0.01-15% by weight of the total composition of a suspending agent.

12. The composition of claim 11 wherein the suspending agent is a montmorillonite mineral or associative thickener.

13. The composition of claim 1 further comprising 0.01-10% by weight of the total composition of a silicone glycol copolymer defoaming agent.

14. The composition of claim 1 further comprising 0.1-35% by weight of the total composition of one or more plasticizers.

15. The composition of claim 14 wherein the plasticizer comprises a glyceryl, glycol, or citrate ester.

16. The composition of claim 14 wherein the plasticizers comprises a compound of the general formula: 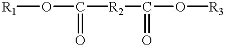
wherein R₁, R₂, and R₃ are each independently a C₁₋₂₀ straight or branched chain alkyl or alkylene which may be substituted with one or more hydroxyl groups.

17. A two container kit for polishing nails comprising:
(a) a first container containing a nail enamel composition comprising, by weight of the total composition:
10-95% solvent, and
5-95% of a film forming polymer having a glass transition temperature in the range of 5 to 90° C. and containing 2 to 29% by weight of the total polymer of at least one polar monomer; and
(b) a second container containing a nail enamel topcoat composition comprising, by weight of the total topcoat composition:
1-99% solvent, and
1-99% of a film forming polymer.

18. The kit of claim 17 wherein the film forming polymer comprises a cellulosic based film former.

19. A method for polishing the nails comprising:
(a) applying to the nails a first composition comprising, by weight of the total composition:
10-95% solvent, and
5-95% of a film forming polymer having a glass transition temperature in the range of 5 to 90° C. and containing about 2 to 29% by weight of the total polymer of at least one polar monomer;
(b) applying to the nails a second composition comprising, by weight of the total composition:
1-99% solvent, and
1-99% of an film forming polymer.

20. The method of claim 19 wherein the dried film formed by (a) and (b) resides on the nails for five to ten days.
